(19) United States
(12) Patent Application Publication
(10) Pub. No.: US 2025/0000378 A1
(43) Pub. Date: Jan. 2, 2025

(54) SYSTEMS AND METHODS FOR MONITORING WITH A HANDHELD CONTROLLER

(71) Applicant: Meta Platforms Technologies, LLC, Menlo Park, CA (US)

(72) Inventors: Xiyu Duan, Sammamish, WA (US); Jianwei Zhang, Westborough, MA (US); Benjamin Kurt Wilson, Snoqualmie, WA (US); Inigo Arnaiz Duenas, Snohomish, WA (US); Travis Taylor, San Jose, CA (US); Chung-Che Huang, Cupertino, CA (US)

(21) Appl. No.: 18/755,099

(22) Filed: Jun. 26, 2024

Related U.S. Application Data
(60) Provisional application No. 63/510,878, filed on Jun. 28, 2023.
(60) Provisional application No. 63/520,389, filed on Aug. 18, 2023.

(51) Int. Cl. A61B 5/024; A61B 5/00; A61B 5/0205; A61B 5/08

(52) U.S. Cl. CPC A61B 5/02433; A61B 5/4266; A61B 5/6897; A61B 5/7214; A61B 5/725; A61B 5/7257; A61B 5/742; A61B 5/0205; A61B 5/0816; A61B 2560/0214; A61B 2560/0223; A61B 2560/0406; A61B 2560/045; A61B 2560/0462; A61B 2562/0219; A61B 2562/0257; A61B 2562/046; A61B 2562/166

(57) ABSTRACT

A method for motion-tolerant optical heart-rate monitoring may include calibrating an array of heart rate sensors of a handheld device by (i) evaluating, while the handheld device is being held by a user, an output of each sensor in the sensor array, (ii) selecting, based on the evaluation of the output of each sensor, a subset of sensors in the sensor array for use in detecting a heart rate of a user, and (iii) using the subset of sensors to monitor the heart rate of the user. Various other methods, systems, and computer-readable media are also disclosed.

### CROSS-REFERENCE TO RELATED APPLICATIONS

This application claims priority to U.S. Application 63/510,878, filed 28 Jun. 2023 and U.S. Application No. 63/520,389, filed 18 Aug. 2023, the disclosures of each of which are incorporated, in their entirety, by this reference.

### BRIEF DESCRIPTION OF THE DRAWINGS

The accompanying drawings illustrate a number of example embodiments and are a part of the specification. Together with the following description, these drawings demonstrate and explain various principles of the present disclosure.

FIG. 1 is an illustration of an example system architecture for motion-tolerant heart rate monitoring featuring a physical connection between a sensor array and a controller.

FIG. 2 is an illustration of example configurations for an array of sensors.

FIG. 3 is a flow diagram of an example method for motion-tolerant heart rate monitoring.

FIG. 4 is an illustration of an example controller integrated with an array of palm-facing sensors.

FIG. 5 is an illustration of an example hand gripping a controller integrated with an array of palm-facing sensors.

FIG. 6 is an illustration of an example controller that includes an array of sensors integrated in a battery cover of the controller.

FIG. 7 is an illustration of an example controller that includes a grip cover integrated with an array of sensors.

FIG. 8 is an illustration of an example palm that includes an array of sensors for heart rate monitoring.

FIG. 9 is an illustration of example output readings for each of the sensors in the array of sensors.

FIG. 10 is an illustration of an example palm that includes an array of sensors during a mid-intensity boxing motion, highlighting a sensor at the upper left corner of the palm.

FIG. 11 is an illustration of an example sensor output reading at the upper left corner of the palm during a mid-intensity boxing motion.

FIG. 12 is an illustration of an example palm that includes an array of sensors during a mid-intensity boxing motion, highlighting a sensor at the bottom of the palm.

FIG. 13 is an illustration of an example sensor output reading at the bottom of the palm during a mid-intensity boxing motion.

FIG. 14 is an illustration of an example palm that includes an array of sensors during a mid-intensity boxing motion, highlighting a sensor at the upper right corner of the palm.

FIG. 15 is an illustration of an example sensor output at the upper right corner of the palm during a mid-intensity boxing motion.

FIGS. 16A and 16B illustrate a handheld controller with monitoring capabilities, such as for fitness data monitoring, according to an additional embodiment of the present disclosure.

FIG. 17 is an exploded perspective view of a portion of a handheld controller, according to at least one embodiment of the present disclosure.

FIG. 18 illustrates a system for monitoring using a handheld controller, such as for monitoring fitness data and/or health data, according to at least one embodiment of the present disclosure.

FIG. 19 is an illustration of an example virtual-reality headset that is used in connection with embodiments of this disclosure.

Throughout the drawings, identical reference characters and descriptions indicate similar, but not necessarily identical, elements. While the example embodiments described herein are susceptible to various modifications and alternative forms, specific embodiments have been shown by way of example in the drawings and will be described in detail herein. However, the example embodiments described herein are not intended to be limited to the particular forms disclosed. Rather, the present disclosure covers all modifications, equivalents, and alternatives falling within the scope of the appended claims.

### DETAILED DESCRIPTION OF EXAMPLE EMBODIMENTS

Many users currently track their personal health and fitness goals by tracking their heart rate daily. It is currently convenient to track a user's heart rate using a smartwatch with an integrated heart rate monitor. However, the lower perfusion within a wrist, where the smartwatch is worn, does not always lend itself to the most accurate heart rate reading. Therefore, it may be advantageous to use a virtual reality/artificial reality (VR/AR) controller held in the palm for heart rate monitoring, taking advantage of the fact that the palm has a higher perfusion than the wrist.

The present disclosure is generally directed to a method for motion-tolerant optical heart rate monitoring in VR/AR controllers. The main challenges that come with heart rate monitoring integrated in VR/AR controllers are the changing grip of a user, varying user hand and palm geometries, and constant motion during use of the controller. To address these challenges, the systems described herein may use a PPG sensor array to probe a large area on the palm to dynamically choose the best sensing location on the palm. For example, the systems described herein may recalibrate an array of sensors to find a different sensing location in response to detecting movement or pressure from the controller.

Features from any of the embodiments described herein may be used in combination with one another in accordance with the general principles described herein. These and other embodiments, features, and advantages will be more fully understood upon reading the following detailed description in conjunction with the accompanying drawings and claims.

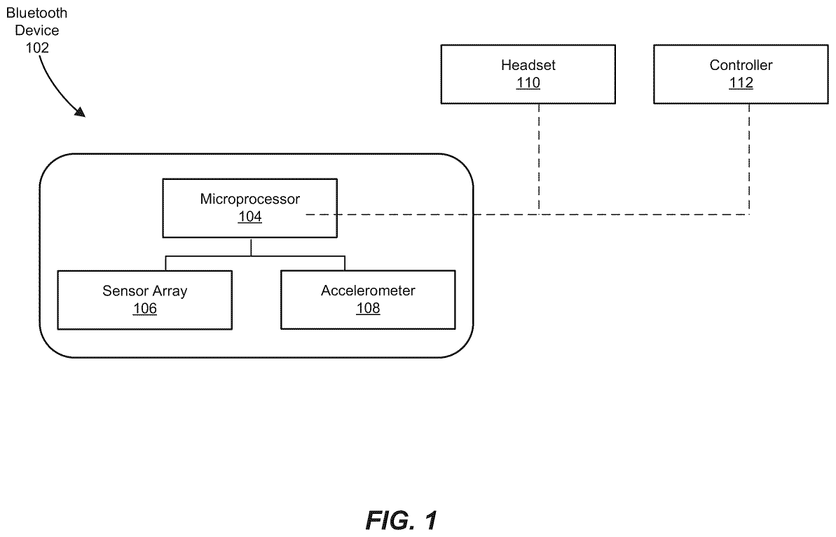

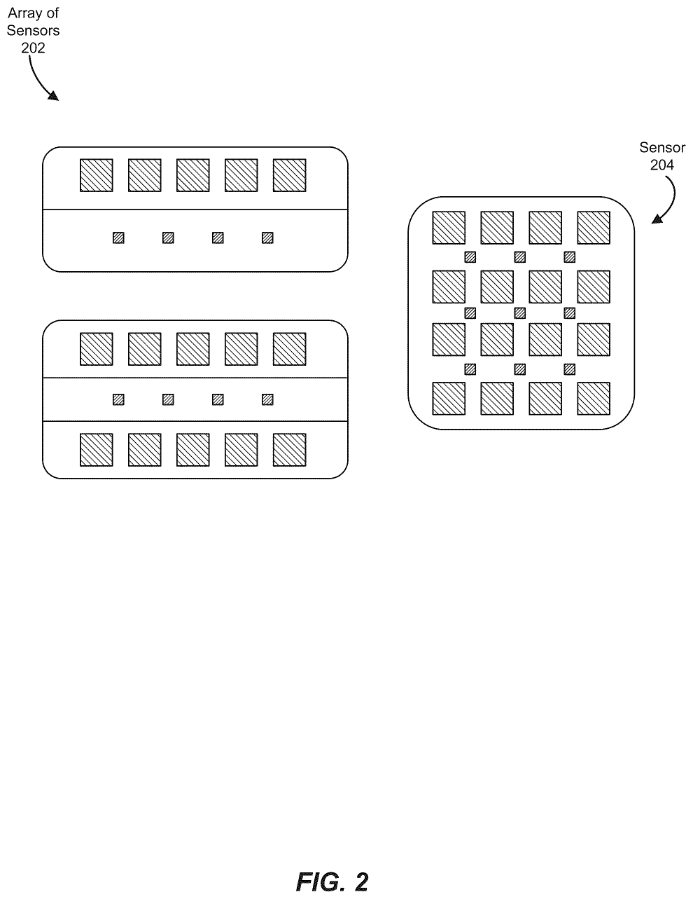

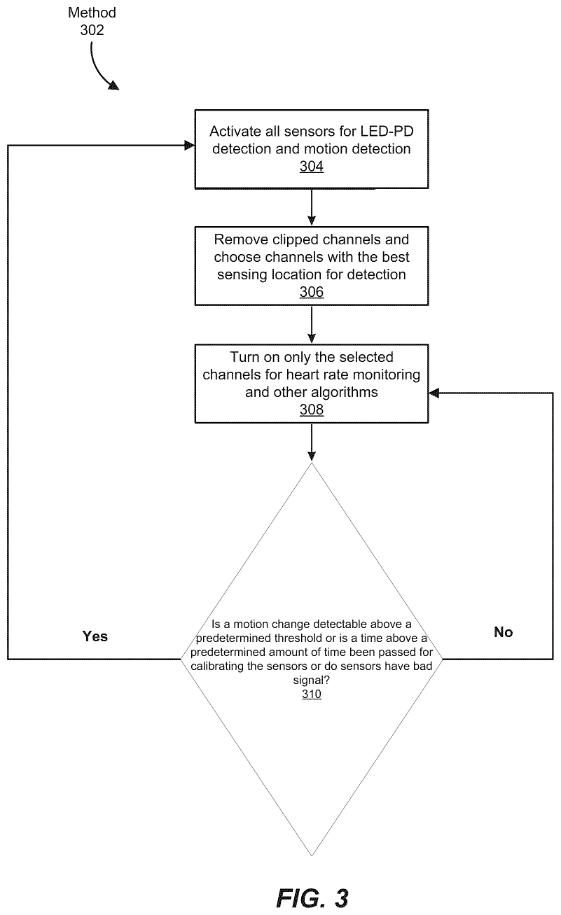

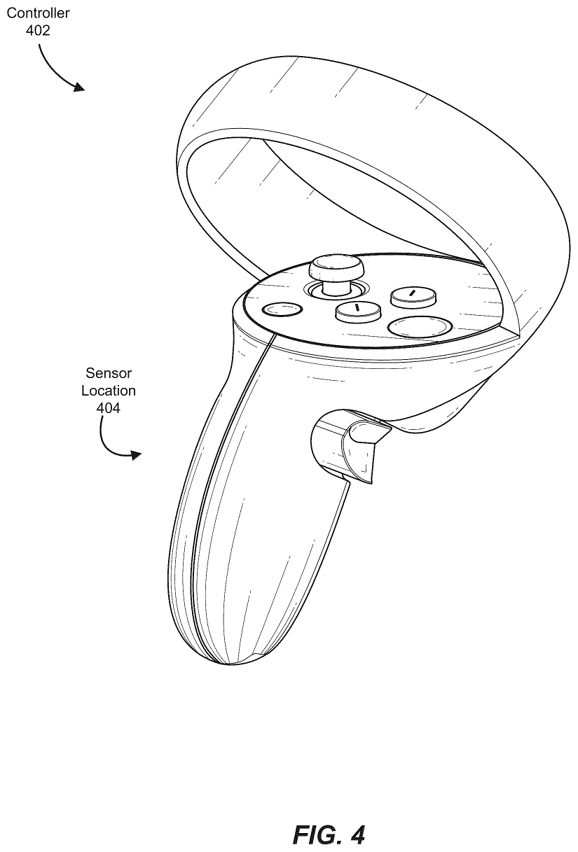

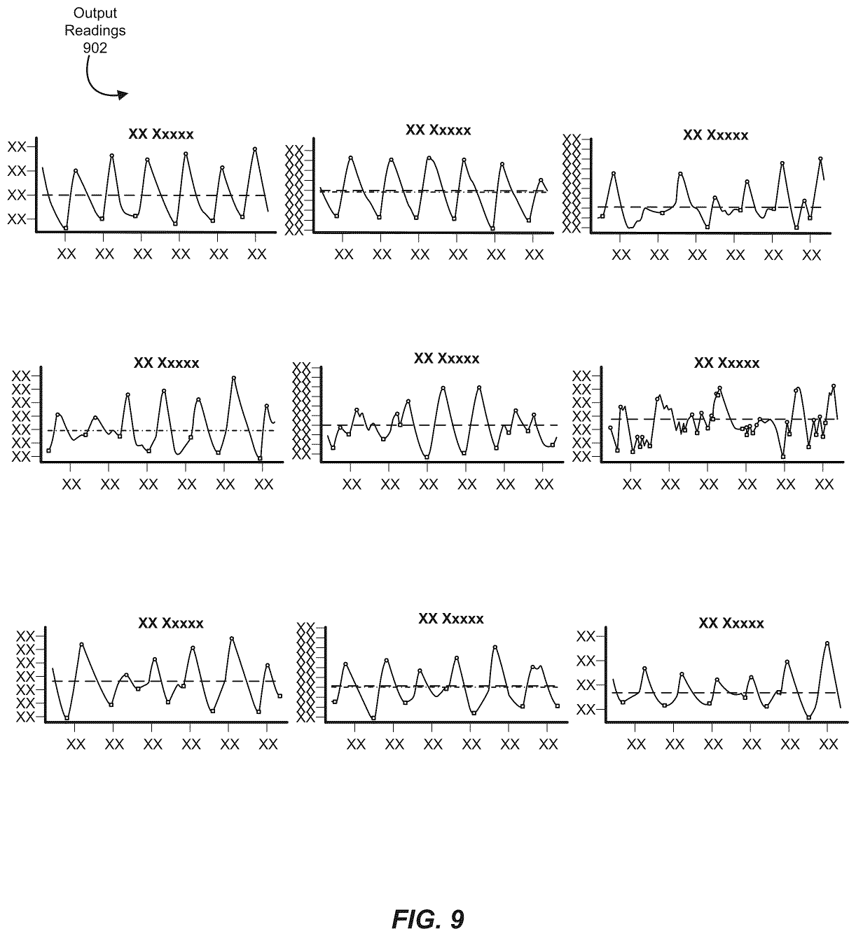

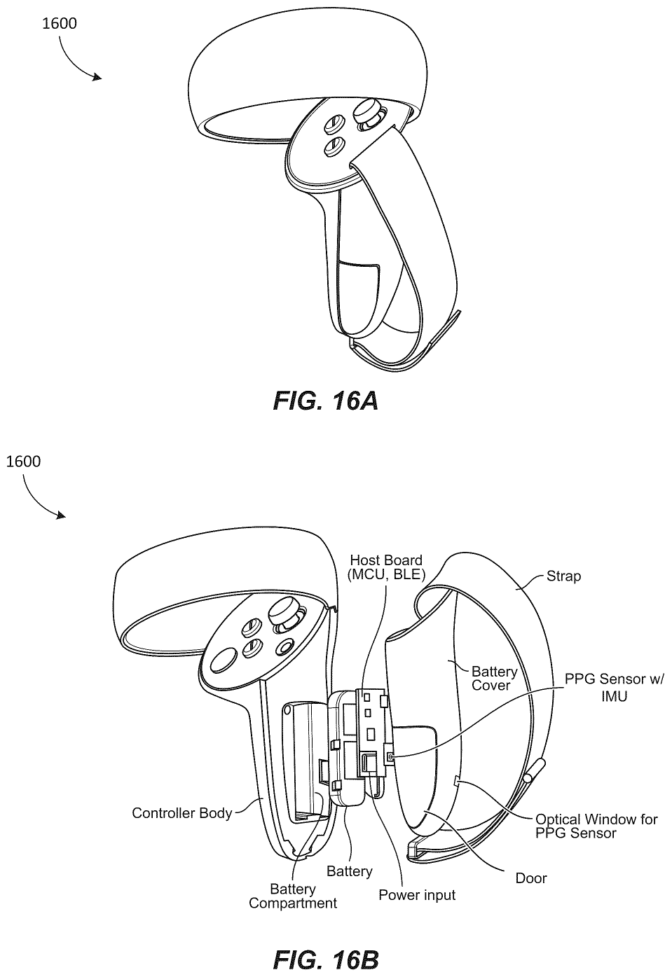

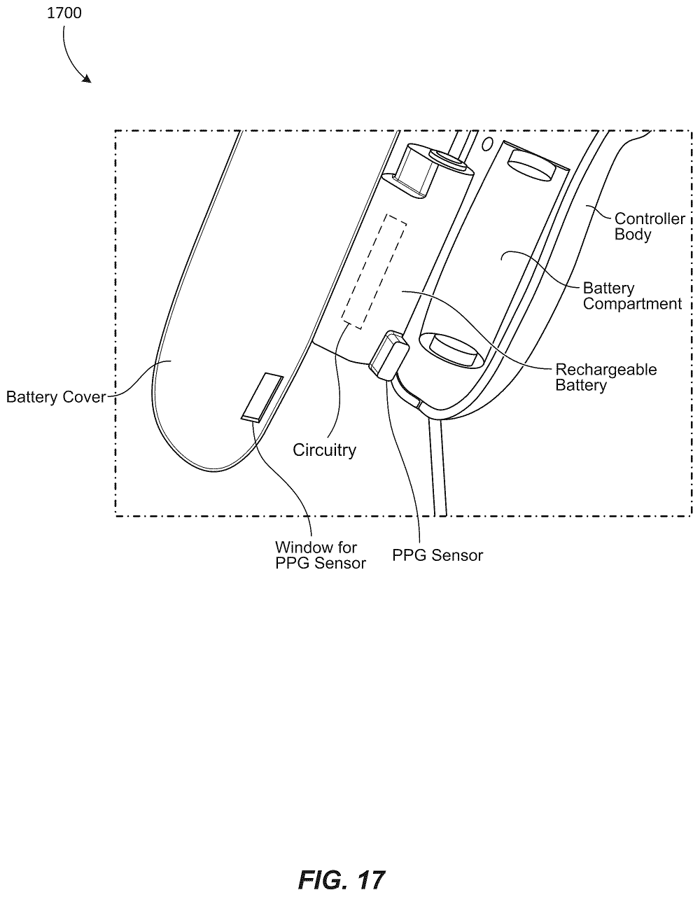

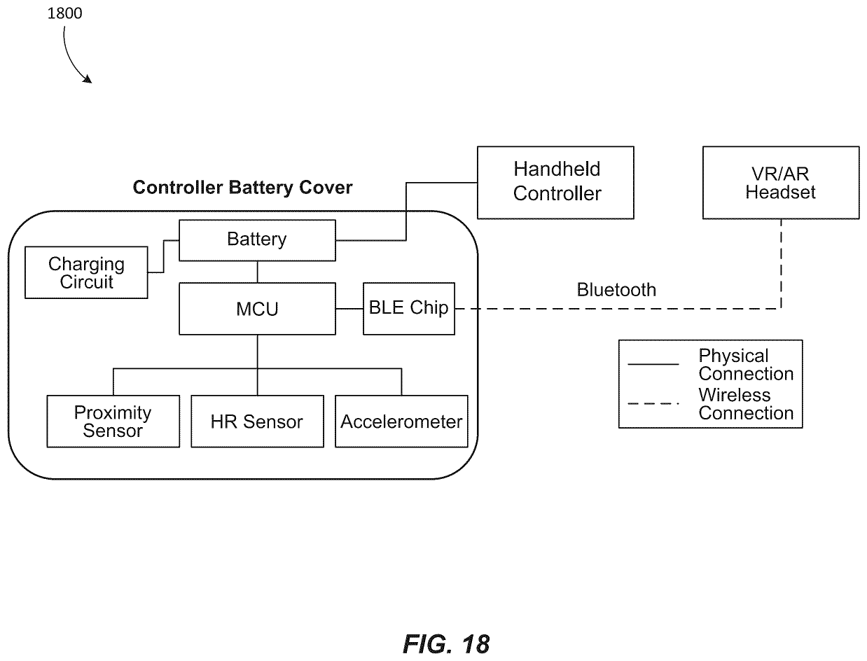

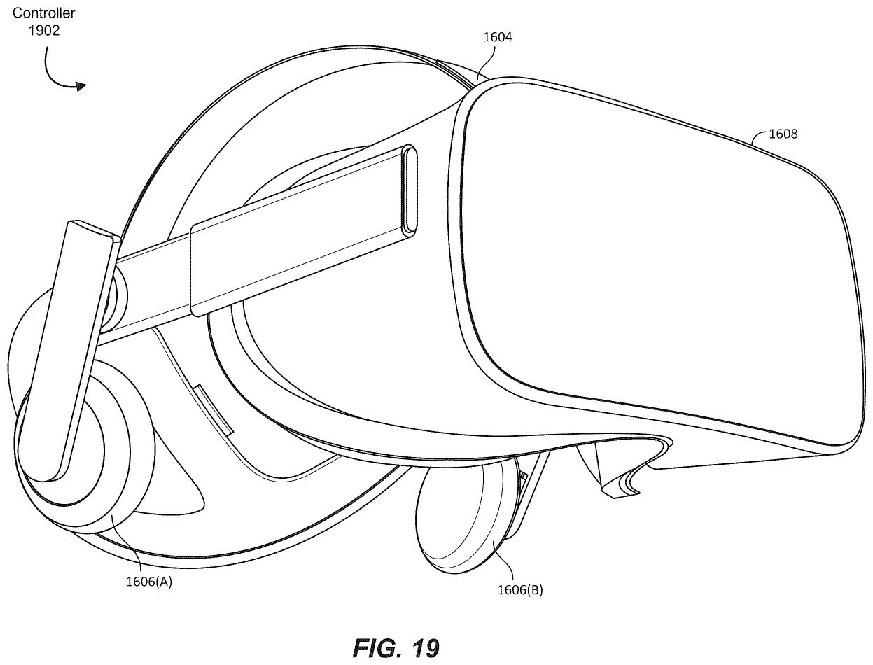

The following will provide, with reference to FIGS. 1-19, detailed descriptions of an integrated PPG sensor array in a VR/AR controller for motion-tolerant heart rate monitoring. The discussion corresponding to FIG. 1 includes a description of an example system architecture featuring communication between the sensors and the controller. The discussion corresponding to FIG. 2 includes a description of example embodiments of a sensor array. The discussion corresponding to FIG. 3 includes a description of a method of motion-tolerant heart rate monitoring in a VR/AR controller. The discussion corresponding to FIGS. 4-8 include various example configurations of palm facing sensors integrated in a controller. The discussion corresponding to FIGS. 9-15 include example output readings of the sensors during mid-level intensity exercise. The discussion corresponding to FIGS. 16A and 16B include a description of an example handheld controller with monitoring capabilities, such as for fitness data monitoring. The discussion corresponding to FIG. 17 includes a description of an example exploded perspective view of a portion of a handheld controller. The discussion corresponding to FIG. 18 includes a description of an example system for monitoring using a handheld controller, such as for monitoring fitness data and/or health data, according to at least one embodiment of the present disclosure. The discussion corresponding to FIG. 19 includes a description of an example virtual-reality headset that may include PPG sensor arrays.

FIG. 1 is an illustration of an example system architecture for motion-tolerant heart rate monitoring featuring a physical connection between a sensor array and a controller. Bluetooth device 102 harbors the communication between a sensor array 106 and a controller 112. The term “controller” may refer to controllers that assist users to perform certain actions in a virtual world. Examples of Bluetooth devices may include, without limitation, sensors, remotes, fitness trackers etc. Bluetooth device 102 may include a microprocessor 104, sensor array 106, and an accelerometer 108. According to some embodiments, the physical connection between Bluetooth device 102 and controller 112 may represent integration of the Bluetooth device 102 within the controller 112. In one embodiment, the physical connection as shown in FIG. 1 may represent the integration of the Bluetooth device 102 in the headset 110.

FIG. 2 is an illustration of the example configurations for an array of sensors in a controller. For example, the systems described here may represent an array of sensors 202 by three different embodiments of sensors 204 as shown in FIG. 2. The term “sensor” may generally refer to a device that detects and measure a heart or a pulse rate for heart rate monitoring. Examples of sensors for heart rate monitoring may include, without limitation, electrical sensors known as electrocardiography (ECG), or optical sensors known as photoplethysmography (PPG). In one embodiment, PPG sensors may include light emitting diodes (LEDs) to measure volumetric variations of blood circulation within the skin. Sensors 204 may represent three different embodiments to make up an array of sensors 202. For example, sensors 204 may include a 1×5 array, 2×5 array, 4×4 array, etc.

FIG. 3 is a flow diagram of an example method 302 for motion-tolerant heart rate monitoring. In some embodiments, the steps shown here may perform any suitable computer-executable code and/or computing system. In one example, each of the steps shown in FIG. 3 may represent an algorithm whose structure includes and/or is represented by multiple sub-steps, examples of which will be provided in greater detail below.

Method 302 includes several steps involved in a motion-tolerant heart rate monitoring process. As illustrated in FIG. 3, one or more of the systems described herein may determine the best sensing location for heart rate monitoring. For example, one or more of the systems described here may select the most optimal sensor in an array of sensors. As noted above, the sensor array may be part of a controller or a headset for heart rate monitoring.

At step 304, the systems described herein may activate all sensor locations for LED-PD detection and motion detection. For example, the array of sensors may activate to determine the best sensing location by calibrating PPG sensors to detect a user's heart rate at a location that may provide the best signal. In some embodiments, PPG sensors in a PPG sensor array may activate in response to detecting external motion from a controller.

Furthermore, as the controller is held by the user, an evaluation on the output of each sensor for signal quality may be done. At step 306, the systems described herein may turn off selected channels for the sensors that may not be in a location suitable for sensing based on the strength of the signal quality. The systems described herein may select the channels for a subset of sensors in the most suitable locations based on evaluating the outputs of the signals for the remaining sensors left in the array. At step 308, the channels for the selected subset of sensors may turn on for heart rate monitoring. In some embodiments, algorithms that may calibrate for specific motions, (based on a pattern, program, etc.) turn on for heart rate monitoring. In some embodiments, calibration of the array of sensors may be response to detecting a reduction in signal quality from at least one sensor.

At step 310, the array of sensors may recalibrate due to a variety of factors that may be present during heart rate monitoring. For example, a change in motion above a predetermined threshold during heart rate monitoring may prompt a recalibration of the sensors in response to the detected movement. In one embodiment, the sensors may recalibrate to determine a new sensing location with better signal quality, away from the detected movement. In further embodiments, at step 310, the array of sensors may recalibrate in response to determining that a predetermined amount of time has been passed since a previous calibration of the sensors. In some embodiments, at step 310, the array of heart sensors may recalibrate in response to poor signal quality for heart rate monitoring.

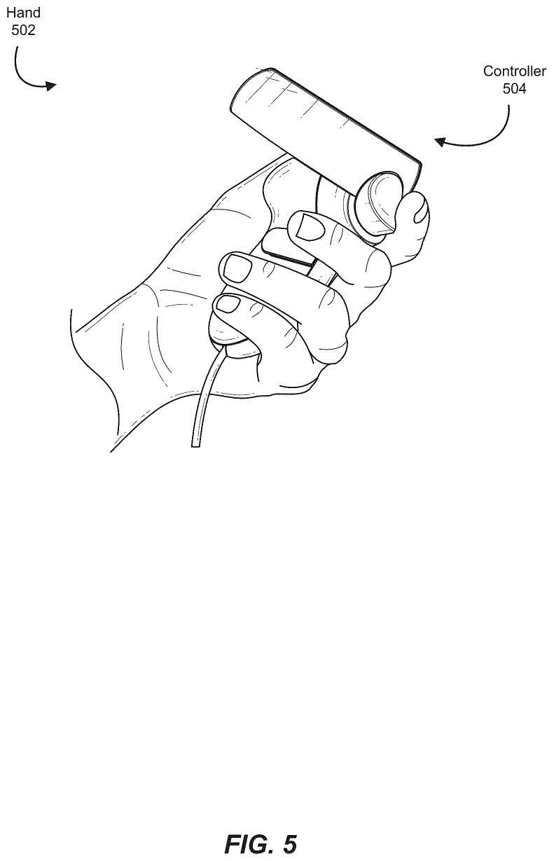

FIG. 4 is an illustration of an example controller integrated with heart rate sensors that may be palm facing. In some embodiments, the controller 402 may include the heart rate sensors natively at a sensor location 404. Sensors at sensor location 404 may integrate in the controller palm facing to conveniently detect a user's grip for heart rate monitoring. FIG. 5 is an illustration of an example hand of a user gripping a controller integrated with palm facing sensors. Controller 504 may include the heart rate sensors natively, where the sensors are facing the palm of a hand 502. Hand 502 may position itself to controller 504, where the array of sensors integrated within controller 504 span across a hand 502.

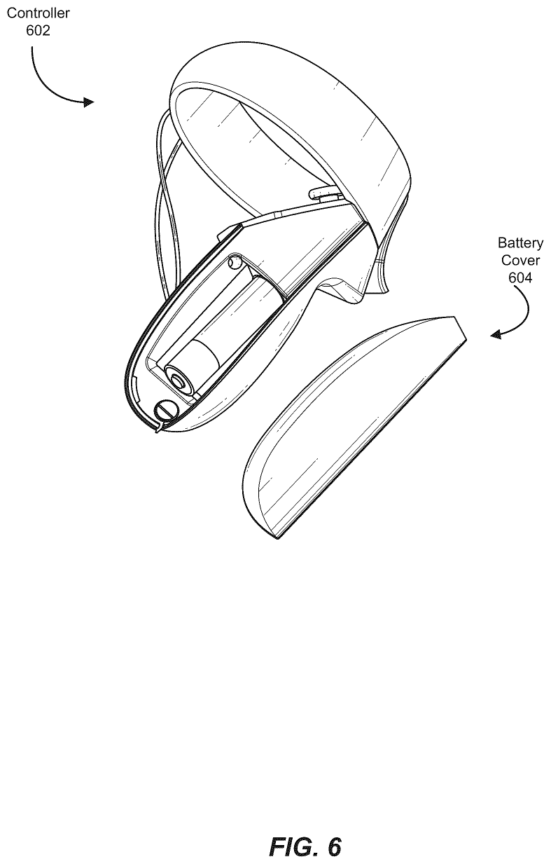

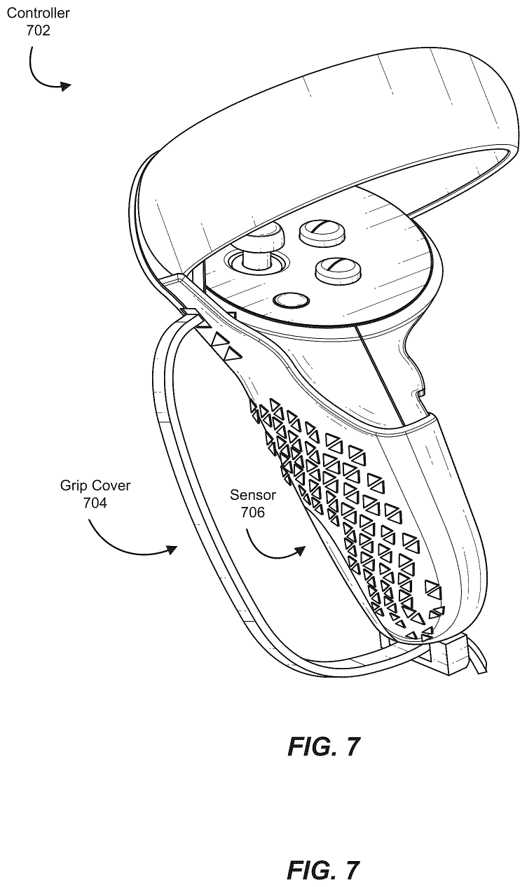

FIG. 6 is an illustration of an example controller integrated with heart rate sensors in a battery cover that may be palm facing. For example, controller 602 may include a battery cover 604 integrated with heart rate sensors that are palm facing. FIG. 7 is an illustration of an example controller with heart rate sensors integrated in a grip cover for the controller. In some embodiments, controller 702 may include a grip cover 704 that has heart rate sensors 706 integrated in the grip cover 704. In further embodiments, grip cover 704 may include heart rate sensors 706 in the areas of controller 702 that a hand may grip for greater detectability.

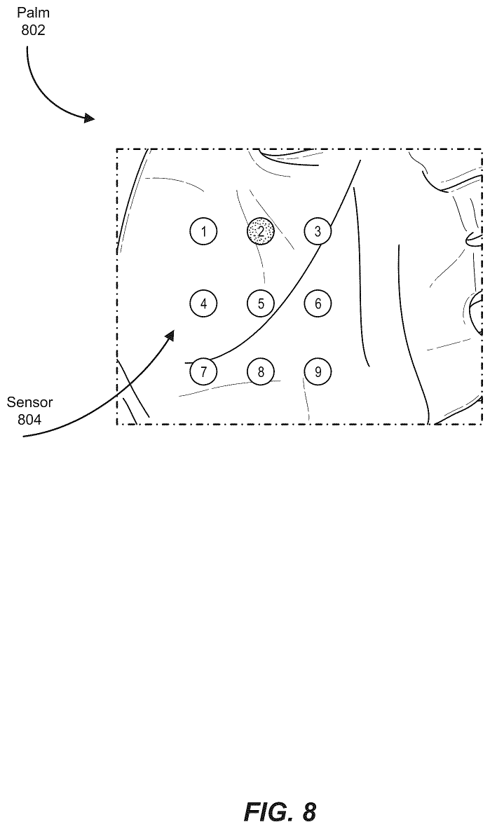

FIG. 8 is an illustration of an example palm of a user including an array of sensors for heart rate monitoring. Sensors 804 may span across a palm 802 of the user to evaluate which sensor may have the best signal. In some embodiments, during calibration of the sensors 804, the user may perform movements instructed to determine the optimal sensing location. As shown earlier in FIG. 3, upon evaluating the output readings of the sensors, the channels of the sensors with the best signal quality are turned on for heart rate monitoring. In some embodiments, sensors 804 may be fit to span across different palm geometries of a user to ensure the most accurate heart rate monitoring regardless of the length, width, or size of the palm.

FIG. 9 is an illustration of example output readings for heart rate sensors. Output readings 902 may include the reading for each of the sensors from the array that detect a user's grip. In some embodiments, the output readings 902 may evaluate to determine which sensor has the strongest signal quality for heart rate monitoring. For example, sensor 2 may have the strongest signal out of output readings 902 making it a candidate for heart rate monitoring on that location of a user's palm.

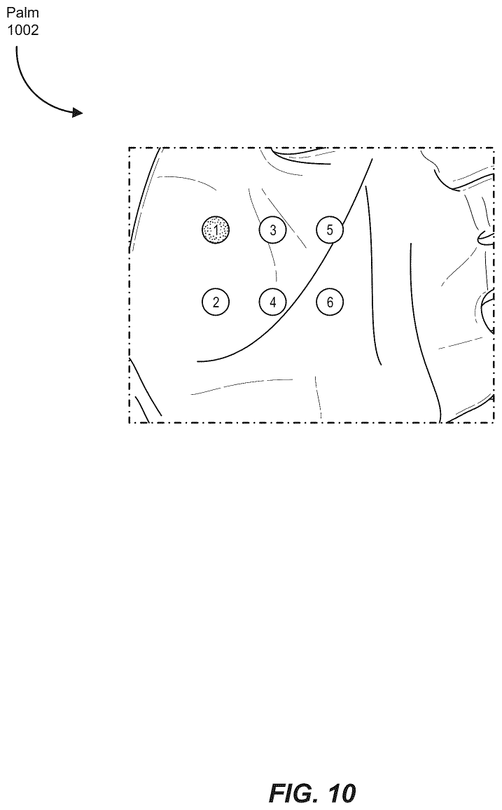

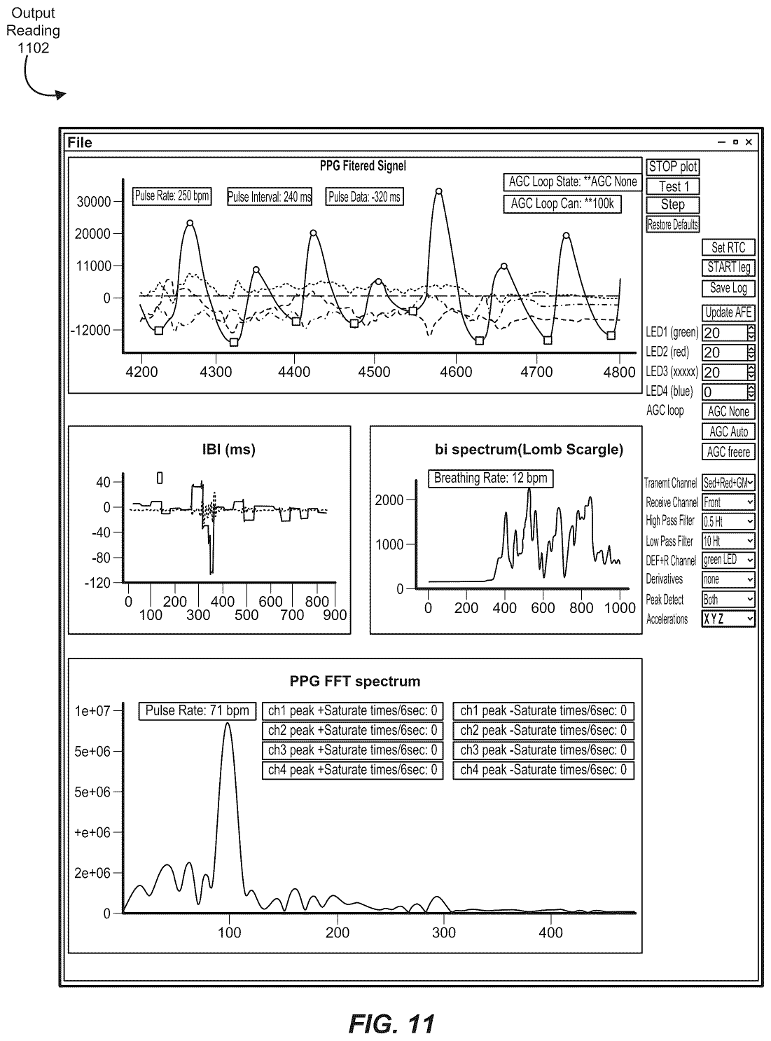

FIG. 10 is an illustration of an example palm of a user including an array of heart rate sensors during a mid-intensity boxing motion, highlighting a sensor at an upper left corner of the palm. Palm 1002 may include an array of heart rate sensors, where each sensor is evaluated to determine the best sensing location for heart rate monitoring during boxing. FIG. 11 is an illustration of the output reading 1102 of a sensor at the upper left corner of the palm. Output reading 1102 may detail the strength of the PPG signal as well as the pulse rate of the user. In some embodiments, this sensor may compare its output reading against other sensors to determine the best signal quality.

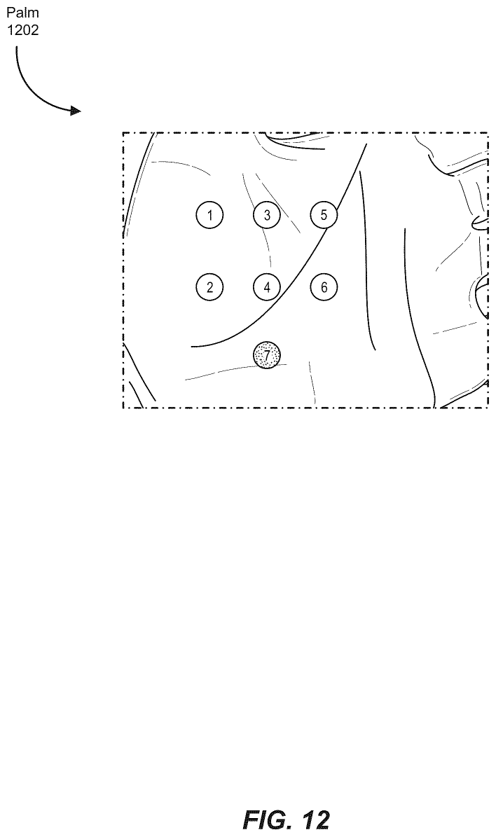

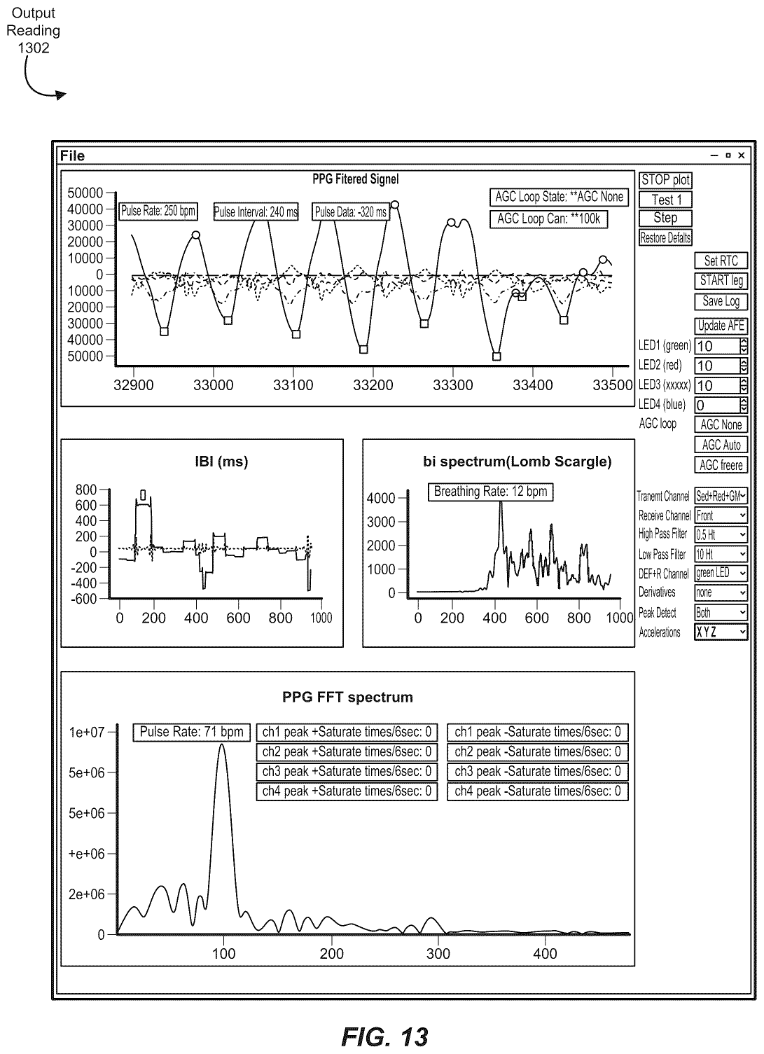

FIG. 12 is an illustration of an example palm of a user including an array of heart rate sensors during a mid-intensity boxing motion, highlighting a sensor at the bottom of the palm. Palm 1202 may include an array of heart rate sensors, where each sensor is evaluated to determine the best sensing location for heart rate monitoring during boxing. FIG. 13 is an illustration of the output reading 1302 of a sensor at the bottom of the palm. Output reading 1302 may detail the strength of the PPG signal as well as the pulse rate of the user. In some embodiments, the sensor at the bottom of the palm may compare its output reading against other sensors to determine the best signal quality.

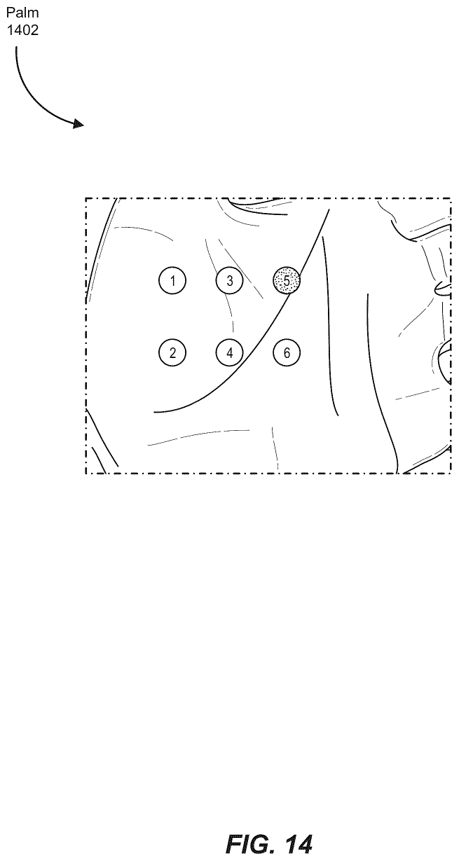

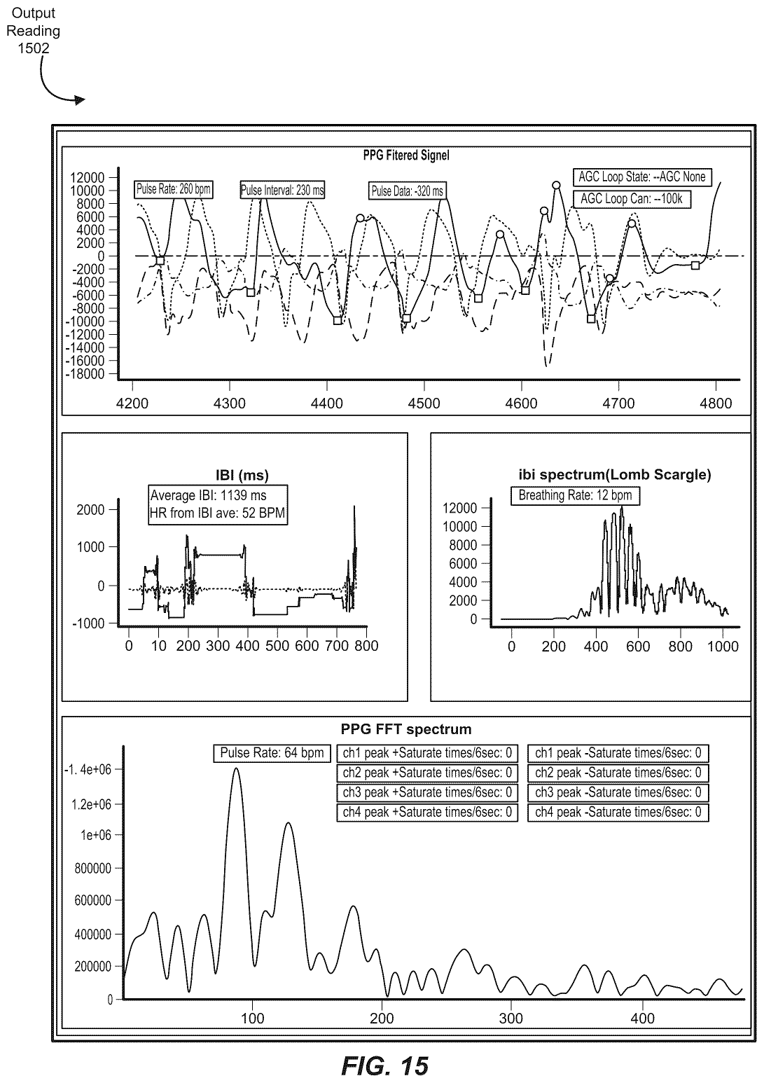

FIG. 14 is an illustration of an example palm of a user including an array of heart rate sensors during a mid-intensity boxing motion, highlighting a sensor at the upper right corner of the palm. Palm 1402 may include an array of heart rate sensors, where each sensor is evaluated to determine the best sensing location for heart rate monitoring during boxing. FIG. 15 is an illustration of the output reading 1502 of a sensor at the upper right corner of the palm. Output reading 1502 may detail the strength of the PPG signal as well as the pulse rate of the user. In some embodiments, the sensor at the upper right corner of the palm may compare its output reading against other sensors to determine the best signal quality.

Handheld controllers may be configured to have monitoring capabilities, such as for fitness tracking and health tracking. In some examples, a handheld controller may include a battery unit that includes a battery, one or more sensor elements (e.g., heart rate monitor, motion sensor, perspiration sensor, etc.), circuitry, and a battery cover with a window for the sensor element to have optical access to a user's hand. In some examples, a battery cover and battery unit may be provided separately, for example as an accessory and/or part replacement for an existing controller. Such controllers may be useful for certain applications, such as artificial-reality applications focusing on fitness (e.g., movement, dancing, punching, exercising, etc.) and wellness (e.g., meditation, mindfulness, yoga, etc.). Compared to relying on a smartwatch or separate fitness tracker, including an integrated heart rate monitor (or other sensor) in an artificial-reality controller may provide a consistent user experience across multiple users and/or across multiple activities for a single user. The performance of the monitor may also be improved over wrist-worn devices since the palm usually exhibits a higher perfusion and lower melanin content than the wrist.

A smart battery cover may be used to upgrade an existing controller with new functionalities. In addition, the battery cover may include an integrated fitness tracker that broadcasts health and fitness metrics, such as the heart rate, through wireless communication protocols (e.g., Bluetooth), to another device or system. For example, the health and fitness metrics may be transmitted with a wireless communication element to the user's smartphone, personal computer, tablet, and/or to a cloud-based database.

The battery cover may include an integrated battery that powers the fitness tracker as well as the controller. The battery cover may be configured to communicate with the controller and/or an associated artificial-reality headset. In some examples, the battery cover may include a proximity sensor that can be used as a power saving mechanism for both the monitoring sensors and the controller (e.g., by starting or stopping operation based on an output of the proximity sensor).

FIGS. 16A and 16B illustrate a handheld controller 1600 (e.g., for an artificial reality system) with monitoring capabilities, such as for fitness data monitoring, according to an additional embodiment of the present disclosure. FIG. 16A is a perspective view of the handheld controller in an assembled state, and FIG. 16B is an exploded perspective view of the handheld controller.

The handheld controller 1600 may include a controller body and a battery cover. The controller body may include a battery compartment for housing a battery (e.g., a rechargeable battery). A strap may be coupled to the battery cover for wrapping around a user's hand when the handheld controller 1600 is in use. A host board may include circuitry for operation of a sensor, such as a photoplethysmography (PPG) sensor, such as for heart rate monitoring, a sweat sensor configured to sense sweat from the user's hand holding the handheld controller, an inertial measurement unit (IMU), and/or other monitoring sensors (e.g., for fitness monitoring and/or health monitoring, etc.). The host board may also include circuitry for charging and recharging the battery. The host board may include a printed circuit board for mounting a microcontroller (MCU), a wireless communication element (e.g., for Bluetooth communication, BLE), memory, a proximity sensor, a power input, and other elements for operation of the handheld controller 1600, the battery, the sensor(s), etc.

The battery cover may include an optical window for the PPG sensor. For example, the optical window may include an aperture through the battery cover. The aperture may be a physical aperture, or the aperture may be a portion of the battery cover that is transparent to light used by the PPG sensor (e.g., infrared light). In addition, the aperture may be covered by a transparent (e.g., transparent to visible light and/or to infrared light) covering, such as clear plastic or glass.

The host board, the rechargeable battery, and the strap may be mounted to the battery cover, such that the host board, rechargeable battery, strap, and battery cover may form a unit that is detachable from the remainder of the handheld controller 1600.

In some examples, the battery cover may also include a door covering an opening. The door may be movable between a closed position (shown in FIGS. 16A and 16B) and an open position that exposes the power input on the host board for charging the rechargeable battery.

FIG. 17 is an exploded perspective view of a portion of a handheld controller 1700, according to at least one embodiment of the present disclosure. The handheld controller 1700 may include a controller body that includes a battery compartment for housing a battery (e.g., a rechargeable battery). A sensor (e.g., PPG sensor) and associated circuitry may be coupled to the battery. The PPG sensor, circuitry, and battery may be included in a single removable unit, as illustrated in FIG. 17. In additional examples, the battery may be a separate unit from the PPG sensor and associated circuitry. A battery cover may include an aperture (e.g., a window) for the PPG sensor so the PPG sensor can have optical access to a user's hand when the handheld controller 1700 is in use. In other words, the PPG sensor may be positioned under the battery cover in a location to sense an input (e.g., data representative of a heart rate) through the aperture.

In additional embodiments, one or more of the PPG sensor, circuitry, and/or associated window for the PPG sensor may be included in the controller body rather than in the battery cover.

FIG. 18 illustrates a system 1800 for monitoring using a handheld controller, such as for monitoring fitness data and/or health data, according to at least one embodiment of the present disclosure.

Artificial reality systems may include a variety of types of visual feedback mechanisms. For example, display devices in augmented-reality system and/or virtual-reality system may include one or more liquid crystal displays (LCDs), light emitting diode (LED) displays, micro LED displays, organic LED (OLED) displays, digital light project (DLP) micro-displays, liquid crystal on silicon (LCoS) micro-displays, and/or any other suitable type of display screen. These artificial reality systems may include a single display screen for both eyes or may provide a display screen for each eye, which may allow for additional flexibility for varifocal adjustments or for correcting a user's refractive error. Some of these artificial reality systems may also include optical subsystems having one or more lenses (e.g., concave or convex lenses, Fresnel lenses, adjustable liquid lenses, etc.) through which a user may view a display screen. These optical subsystems may serve a variety of purposes, including to collimate (e.g., make an object appear at a greater distance than its physical distance), to magnify (e.g., make an object appear larger than its actual size), and/or to relay (to, e.g., the viewer's eyes) light. These optical subsystems may be used in a non-pupil-forming architecture (such as a single lens configuration that directly collimates light but results in so-called pincushion distortion) and/or a pupil-forming architecture (such as a multi-lens configuration that produces so-called barrel distortion to nullify pincushion distortion).

In addition to or instead of using display screens, some of the artificial reality systems described herein may include one or more projection systems. For example, display devices in an augmented-reality system and/or a virtual-reality system may include micro-LED projectors that project light (using, e.g., a waveguide) into display devices, such as clear combiner lenses that allow ambient light to pass through. The display devices may refract the projected light toward a user's pupil and may enable a user to simultaneously view both artificial reality content and the real world. The display devices may accomplish this using any of a variety of different optical components, including waveguide components (e.g., holographic, planar, diffractive, polarized, and/or reflective waveguide elements), light-manipulation surfaces and elements (such as diffractive, reflective, and refractive elements and gratings), coupling elements, etc. Artificial reality systems may also be configured with any other suitable type or form of image projection system, such as retinal projectors used in virtual retina displays.

Some artificial reality systems may, instead of blending an artificial reality with actual reality, substantially replace one or more of a user's sensory perceptions of the real world with a virtual experience. One example of this type of system is a head-worn display system, such as virtual-reality system 1902 in FIG. 19 that mostly or completely covers a user's field of view. Virtual-reality system 1902 may include a front rigid body 1908 and a band 1904 shaped to fit around a user's head. Virtual-reality system 1902 may also include output audio transducers 1906(A) and 1906(B). Furthermore, while not shown in FIG. 19, front rigid body 1908 may include one or more electronic elements, including one or more electronic displays, one or more inertial measurement units (IMUs), one or more tracking emitters or detectors, and/or any other suitable device or system for creating an artificial-reality experience.

The artificial reality systems described herein may also include various types of computer vision components and subsystems. For example, augmented-reality system and/or virtual-reality system 1902 may include one or more optical sensors, such as two-dimensional (2D) or 3D cameras, structured light transmitters and detectors, time-of-flight depth sensors, single-beam or sweeping laser rangefinders, 3D LiDAR sensors, and/or any other suitable type or form of optical sensor. An artificial reality system may process data from one or more of these sensors to identify a location of a user, to map the real world, to provide a user with context about real-world surroundings, and/or to perform a variety of other functions.

The artificial reality systems described herein may also include one or more input and/or output audio transducers. Output audio transducers may include voice coil speakers, ribbon speakers, electrostatic speakers, piezoelectric speakers, bone conduction transducers, cartilage conduction transducers, tragus-vibration transducers, and/or any other suitable type or form of audio transducer. Similarly, input audio transducers may include condenser microphones, dynamic microphones, ribbon microphones, and/or any other type or form of input transducer. In some embodiments, a single transducer may be used for both audio input and audio output.

In some embodiments, the artificial reality systems described herein may also include tactile (i.e., haptic) feedback systems, which may be incorporated into headwear, gloves, body suits, handheld controllers, environmental devices (e.g., chairs, floormats, etc.), and/or any other type of device or system. Haptic feedback systems may provide various types of cutaneous feedback, including vibration, force, traction, texture, and/or temperature. Haptic feedback systems may also provide various types of kinesthetic feedback, such as motion and compliance. Haptic feedback may be implemented using motors, piezoelectric actuators, fluidic systems, and/or a variety of other types of feedback mechanisms. Haptic feedback systems may be implemented independent of other artificial reality devices, within other artificial reality devices, and/or in conjunction with other artificial reality devices.

By providing haptic sensations, audible content, and/or visual content, artificial reality systems may create an entire virtual experience or enhance a user's real-world experience in a variety of contexts and environments. For instance, artificial reality systems may assist or extend a user's perception, memory, or cognition within a particular environment. Some systems may enhance a user's interactions with other people in the real world or may enable more immersive interactions with other people in a virtual world. Artificial reality systems may also be used for educational purposes (e.g., for teaching or training in schools, hospitals, government organizations, military organizations, business enterprises, etc.), entertainment purposes (e.g., for playing video games, listening to music, watching video content, etc.), and/or for accessibility purposes (e.g., as hearing aids, visual aids, etc.). The embodiments disclosed herein may enable or enhance a user's artificial reality experience in one or more of these contexts and environments and/or in other contexts and environments.

A PPG sensor array may be used for heart rate monitoring by integrating the PPG sensor array into a VR controller. A PPG sensor array may dynamically choose the best sensing location considering factors such as motion and pressure that may impair signal quality. An array of sensors may allow for greater signal quality to be achieved compared to a single sensor due to varying palm geometries and grip movement changing when using a VR controller that a single sensor may not be able to capture. An algorithm may be designed to choose a sensor within the array that is most optimal for the best signal quality given the factors present. For example, during the initiation phase, the user may be instructed to perform certain movements. In this phase, all channels may be enabled to determine the most optimal sensing location. In various embodiments, multiple sensors in the PPG sensor array may be used for heart rate monitoring. Furthermore, a PPG sensor array may probe a large area on a palm that may provide more accurate heart rate tracking than a traditional smart watch worn on a wrist. Accordingly, the PPG sensor array integrated into the VR controller may provide for a more consistent user experience.

As detailed above, the computing devices and systems described and/or illustrated herein broadly represent any type or form of computing device or system capable of executing computer-readable instructions, such as those contained within the modules described herein. In their most basic configuration, these computing device(s) may each include at least one memory device and at least one physical processor.

In some examples, the term “memory” or “memory device” generally refers to any type or form of volatile or non-volatile storage device or medium capable of storing data and/or computer-readable instructions. In one example, a memory device may store, load, and/or maintain one or more of the modules described herein. Examples of memory devices include, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In some examples, the term “physical processor” generally refers to any type or form of hardware-implemented processing unit capable of interpreting and/or executing computer-readable instructions. In one example, a physical processor may access and/or modify one or more modules stored in the above-described memory device. Examples of physical processors include, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the modules described and/or illustrated herein may represent portions of a single module or application. In addition, in certain embodiments one or more of these modules may represent one or more software applications or programs that, when executed by a computing device, may cause the computing device to perform one or more tasks. For example, one or more of the modules described and/or illustrated herein may represent modules stored and configured to run on one or more of the computing devices or systems described and/or illustrated herein. One or more of these modules may also represent all or portions of one or more special-purpose computers configured to perform one or more tasks.

In addition, one or more of the modules described herein may transform data, physical devices, and/or representations of physical devices from one form to another. For example, one or more of the modules recited herein may receive [data] to be transformed, transform the [data], output a result of the transformation to [perform a function], use the result of the transformation to [perform a function], and store the result of the transformation to [perform a function]. Additionally or alternatively, one or more of the modules recited herein may transform a processor, volatile memory, non-volatile memory, and/or any other portion of a physical computing device from one form to another by executing on the computing device, storing data on the computing device, and/or otherwise interacting with the computing device.

In some embodiments, the term “computer-readable medium” generally refers to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

The preceding description has been provided to enable others skilled in the art to best utilize various aspects of the example embodiments disclosed herein. This example description is not intended to be exhaustive or to be limited to any precise form disclosed. Many modifications and variations are possible without departing from the spirit and scope of the present disclosure. The embodiments disclosed herein should be considered in all respects illustrative and not restrictive. Reference should be made to the appended claims and their equivalents in determining the scope of the present disclosure.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and have the same meaning as the word “comprising.”

What is claimed is:

1. A method comprising:
calibrating an array of heart rate sensors of a handheld device by:
evaluating, while the handheld device is being held by a user, an output of each sensor in the sensor array;
selecting, based on the evaluation of the output of each sensor, a subset of sensors in the sensor array for use in detecting a heart rate of a user; and
using the subset of sensors to monitor the heart rate of the user.

2. The method of claim 1, wherein the evaluation is based on a strength of a signal quality.

3. The method of claim 1, further comprising calibrating the array of heart rate sensors in response to detecting a reduction signal quality from at least one sensor in the subset of sensors.

4. The method of claim 1, further comprising detecting movement of the handheld device, wherein the calibrating the array of heart rate sensors is performed in response to detecting the movement.

5. The method of claim 1, further comprising determining that a predetermined amount of time has passed since a previous calibration of the array of heart sensors, wherein the calibrating the array of heart rate sensors is performed in response to the determination that the predetermined amount of time has passed.

6. A handheld controller, comprising:
a battery compartment configured for housing a battery to provide power to the handheld controller;
a battery cover configured for covering the battery compartment, the battery cover including an aperture; and
a sensor positioned under the battery cover in a location to sense an input through the aperture.

7. The handheld controller of claim 6, wherein the sensor comprises a sweat sensor configured to sense sweat from a hand of a user holding the handheld controller.

8. The handheld controller of claim 6, wherein the sensor comprises a heart rate sensor configured to sense a heart rate of a user holding the handheld controller from a hand of the user.

9. The handheld controller of claim 6, further comprising a rechargeable battery, wherein the battery compartment is configured for housing the rechargeable battery.

10. The handheld controller of claim 9, wherein the rechargeable battery is coupled to the battery cover.

11. The handheld controller of claim 9, further comprising a printed circuit board coupled to the battery cover, wherein the rechargeable battery is configured to provide power to the printed circuit board and to the handheld controller.

12. The handheld controller of claim 9, wherein the battery cover further comprises at least one door covering an opening, the at least one door movable between a closed position and an open position, wherein a power input for charging the rechargeable battery is positioned behind the at least one door to be exposed through the opening when the door is in the open position.

13. The handheld controller of claim 6, further comprising a printed circuit board coupled to the battery cover, wherein the sensor is mounted to the printed circuit board.

14. The handheld controller of claim 13, further comprising a wireless communication element mounted to the printed circuit board.

15. The handheld controller of claim 6, further comprising a strap coupled to the battery cover and configured to wrap around a user's hand when holding the handheld controller.

16. A battery module for a handheld controller, the battery module comprising:
a battery cover shaped and sized for covering a battery compartment of the handheld controller, the battery cover including an aperture passing through the battery cover;
a sensor coupled to the battery cover and positioned to sense at least one input through the aperture; and
a rechargeable battery coupled to the battery cover, the rechargeable battery configured to provide power to at least the sensor.

17. The battery module of claim 16, further comprising a strap coupled to the battery cover and configured for wrapping around a user's hand.

18. The battery module of claim 16, further comprising a power input for charging the rechargeable battery.

19. The battery module of claim 18, wherein the battery cover further comprises a door covering an opening, wherein the power input is positioned behind the door and is accessible through the opening when the door is in an open position.

20. The battery module of claim 16, further comprising a printed circuit board, wherein the sensor is mounted to the printed circuit board.
